(19) United States
(12) Patent Application Publication
(10) Pub. No.: US 2006/0161226 A1
(43) Pub. Date: Jul. 20, 2006

(54) Apparatus and method for reducing follicular cell apoptosis

(76) Inventor: George R. McMickle, Las Vegas, NV (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/038,779

(22) Filed: Jan. 18, 2005

(51) Int. Cl. A61N 5/06

(52) U.S. Cl. 607/88

(57) ABSTRACT

The invention provides a light emitting device having an array of light emitting diodes (LED). The array of the device is arranged to illuminate a surface area of a scalp and the LEDs exhibit a plurality of wavelengths within the near infrared region of the electromagnetic spectrum between about 600-1000 nm, wherein the plurality of wavelengths synergistically combine to decrease follicular apoptosis. The array of light emitting diodes can include LEDs that are pulsed and can contain an array of light emitting diodes corresponding to about 30-200, preferably about 50-150, and more preferably about 80-120 LEDs. The light emitting device of the invention also can include a headband or a stationary mount or other mount for positioning over a target area. Also provided is a method of reducing follicular cell apoptosis. The method includes illuminating a surface area of a scalp containing a follicular hair cell for an effective period of time with an array of light emitting diodes (LED). The array of LEDs exhibit a plurality of wavelengths within the near infrared region of the electromagnetic spectrum between about 600-100 nm, wherein the plurality of wavelengths synergistically combine to decrease follicular cell apoptosis. The method can include inducing an increase in nitric oxide production compared to an untreated follicular hair cell as well as inducing mitochondrial chromophore activation. An effective period of time can include about 5-30 minutes, about 10-20 minutes or about 12-18 minutes and can promote vaso perfusion of the scalp or reduction in hair loss.

### BACKGROUND OF THE INVENTION

This invention relates generally to cellular apoptosis and, more specifically to the inhibition of follicular cell apoptosis for the treatment of alopecia.

Hair loss affects millions of individuals in the United States and can be the cause of severe anxiety, reduction in self-esteem and overall unhappiness on a daily basis. Hair loss or alopecia affects both men and women and can cause many individuals to substantially modify daily routines or appearances as well as to undergo cosmetic surgical procedures in an attempt to change their outward appearance. For example, many individuals alter their preferred hair style or wear head coverings to reduce the overtness of a balding appearance or to completely conceal the appearance. Such behavioral modifications can lead to undue stress, anxiety or self-esteem. Additionally, those wishing to change their appearance are susceptible to fraud from vendors of products that have little efficacy for reducing alopecia and are nothing more than a placebo marketed under the guise of promoting hair regeneration.

Substantial economic and human resources also have been invested into discovering and developing treatments to prevent hair loss, promote hair regrowth or both. For example, the commercial market for effective treatment is substantial and drugs such as Minoxidil or Rogaine have made entry into the market and purport to regenerate hair growth. However, the effects of these treatments vary and treatment periods are time consuming and cumbersome. Some individuals have reported little or no result after prolonged treatment. Cosmetic surgical procedures aimed at transplanting hair follicles also has been met with similar inconsistencies and prolonged treatments.

Thus, there exists a need for a means and for an effective treatment method that will prevent progression in the severity of alopecia or regrow lost hair. The present invention satisfies this need and provides related advantages as well.

### SUMMARY OF THE INVENTION

The invention provides a light emitting device having an array of light emitting diodes (LED). The array of the device is arranged to illuminate a surface area of a scalp and the LEDs exhibit a plurality of wavelengths within the near infrared region of the electromagnetic spectrum between about 600-1000 nm, wherein the plurality of wavelengths synergistically combine to decrease follicular apoptosis. The array of light emitting diodes can include LEDs that are pulsed and can contain an array of light emitting diodes corresponding to about 30-200, preferably about 50-150, and more preferably about 80-120 LEDs. The light emitting device of the invention also can include a headband or a stationary mount or other mount for positioning over a target area. Also provided is a method of reducing follicular cell apoptosis. The method includes illuminating a surface area of a scalp containing a follicular hair cell for an effective period of time with an array of light emitting diodes (LED). The array of LEDs exhibit a plurality of wavelengths within the near infrared region of the electromagnetic spectrum between about 600-100 nm, wherein the plurality of wavelengths synergistically combine to decrease follicular cell apoptosis. The method can include inducing an increase in nitric oxide production compared to an untreated follicular hair cell as well as inducing mitochondrial chromophore activation. An effective period of time can include about 5-30 minutes, about 10-20 minutes or about 12-18 minutes and can promote vaso perfusion of the scalp or reduction in hair loss.

### BRIEF DESCRIPTION OF THE DRAWINGS

FIG. 1 shows an exemplary circular arrangement of 96 LEDs for a light emitting device suitable for illuminating the scalp for treatment of follicular cell apoptosis and alopecia.

FIG. 2 shows one circuit design for supply power, regulation and wiring for the light emitting device having 96 near infrared LEDs ranging from 680-910 nm which is exemplified in FIG. 1.

FIG. 3 shows a light emitting device of the invention attached to a headband that positions the device above the scalp of a subject. In FIG. 3A, the device is positioned on the subjects head with an array of LEDs in juxtaposition to the scalp. In FIG. 3B, the device is off the subject and the array is tilted upwards for viewing. Adjacent and connected to the array is part of the circuitry and power source.

### DETAILED DESCRIPTION OF THE INVENTION

This invention is directed to a device and a method for reducing follicular cell apoptosis. The device utilizes near infrared light emitting diodes (LED) that produce pulsed radiation in a range of about 600-1000 nm. Reduction in follicular cell apoptosis can promote hair regeneration or hair re-growth as well as simultaneously promote both hair regeneration and hair re-growth. One advantage of exposing hair follicles to near infrared radiation is the induction of two modes of apoptotic inhibitory action. In one mode of action, pulsed radiation in the near infrared range is a potent and long acting vasodilator. Exposure of follicular cells to near infrared radiation results in the production of nitric oxide (NO), facilitating healing of injured or unhealthy cells or tissues. In a second mode of action induced by pulsed radiation in the near infrared range, mitochondrial chromophores are excited, which lead to enhanced cellular energy levels and concomitant cellular regeneration and re-growth. Either or both modes of action inhibit the entry or progression of programmed cell death or apoptosis of follicular cells, resulting in enhanced viability follicle cells and promotion of hair regeneration and/or re-growth.

In one embodiment, the invention is directed to a device having LED arranged to focus near infrared radiation emission onto the scalp. A plurality of LEDs are arranged in a circular or oval array so that near infrared emissions impinge on follicular cells of the scalp. Interspersed subsets of LEDs emit near infrared wavelengths within discrete wavelength ranges such that the collection of LEDs emit a near infrared range spanning 610-980 nm. The LED emissions also can be pulsed at durations of about 85 msec. The device is useful for the treatment of alopecia as well as for promoting regeneration of natural hair color.

In another embodiment, the invention is directed to a method of reducing follicular cell apoptosis. Inhibition of follicular cell apoptosis is accomplished by exposing follicular cells to a plurality of LEDs emitting near infrared radiation. The greater the number of exposures the greater the inhibition of follicular cell apoptosis. Follicular cells are exposed to near infrared radiation within the range of 600-1000 nanometers and preferably exposure of a plurality of discrete wavelengths within and spanning this range is accomplished. The discrete wavelengths can be pulsed and combine to significantly decrease follicular cell apoptosis, thus increasing hair viability and retention.

As used herein, the term “array” is intended to mean an arrangement or group of elements that together form a unit. When used in reference to the light emitting device of the invention, a array includes a group of light emitting diodes that emit near radiation within the near infrared spectrum. An array can be arranged in two-dimensional planer structure, a three dimensional structure or other complex combination of structures so long as the light emitting diode elements form a functional unit.

As used herein, the term “near infrared” is intended to mean shorter wavelengths of radiation in the infrared spectrum between about 600 nm to 1000 nm.

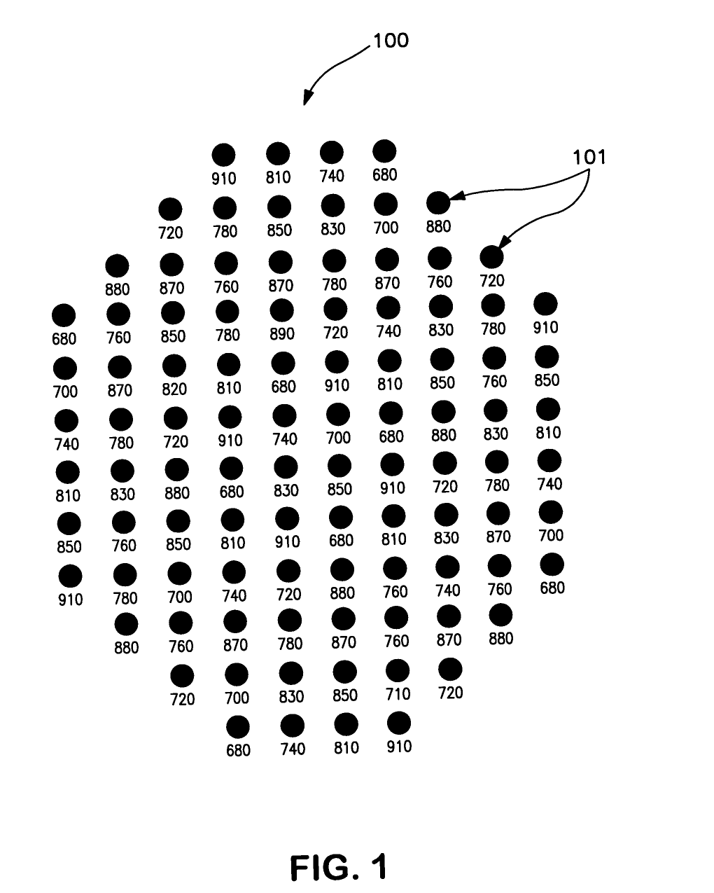

As used herein, the term “plurality” is intended to mean a population of at least two constituents. Generally, a plurality will include 3, 4, 5, 6, 7, 8, 9 or 10 or more constituents. A plurality also can include 15, 20, 25, 50, 75, 100 or more constituents. A plurality similarly can include hundreds or thousands of constituents including 200, 300, 400, 500, 600, 700, 800, 900 or 1000 or more members. The term as it is used herein is intended to include all integer values within the above exemplary sizes. A specific example of a plurality of different wavelengths is the device shown in FIG. 1 where an array of 96 LEDs emit a total of 12 different wavelengths.

As used herein, the term “synergistically” or “synergy” when used in reference to the combined effect different near infrared wavelengths is intended to mean a cooperative action of the discrete wavelengths such that the total effect is greater than the sum of each effect taken independently.

As used herein, the term “apoptosis” is intended to mean programmed cell death, which includes a genetically directed process of cell self-destruction that is marked by the fragmentation of nuclear DNA. Apoptosis can be activated either by the presence or removal of a stimulus and is a normal physiological process eliminating DNA-damaged, superfluous, or unwanted cells, and when inhibited can result in cell proliferation.

The invention provides a light emitting device having an array of light emitting diodes (LED). The array includes and arrangement of the LEDs that illuminate a surface area of a scalp, the LEDs having a plurality of wavelengths within the near infrared region of the electromagnetic spectrum between about 600-1000 nm, wherein the plurality of wavelengths synergistically combine to decrease follicular apoptosis.

Exposure of cells or tissue to near infrared radiation of the electromagnetic spectrum between about 600-1000 nm enhances vaso perfusion and energy levels of the exposed cells or tissue. The light emitting device of the invention is applicable to treat injured or apoptotic cells because enhanced perfusion and energy levels can retard or reverse progression of the injury or apoptotic cellular state. As described further below, the device can be applied to tissues such as the epidermis, including the scalp and hair follicle cells therein, to treat or apoptotic cells. Exemplary applications for the light emitting device of the invention is its use for reducing hair loss, promoting hair regeneration and/or promoting hair re-growth.

A light emitting device of the invention organizes one or more illumination elements to emit one or more wavelengths of radiation within the near infrared region of the electromagnetic spectrum between about 600-1000 nm. The emissions can be produced from any of various illuminating sources including, for example, incandescent, fluorescent or photon producing semiconductor material such as a light emitting diode. Other sources well known in the art that produce radiation within the near infrared region of about 600-1000 nm also can be employed in the light emitting device of the invention. Light sources that can direct and/or focus the emitted radiation are beneficial because they increase the efficiency of exposure to the targeted area and, therefore, are easily calibrated or quantitated.

Light emitting diodes (LED) 101 are particularly suited for use in the light emitting device of the invention because, for example, they direct light in an outward direction and efficiently produce photons with little heat generation. Additional attributes of LEDs useful in the light emitting device of the invention is that LEDs can be miniaturized and routinely fit into an electrical circuit, but unlike incandescent, fluorescent or halogen sources, LEDs do not have a filament or chemical component that will burn out. Rather, LEDs are illuminated solely by the movement of electrons in a semiconductor material. LEDs also exhibit long illumination lifetimes, lasting as long as a standard transistor, and having between about 50,000 to 100,000 hours compared with incandescent bulbs lasting about 1,000 hours or fluorescent sources lasting about 7,500 hours.

A light emittng diode (LED) is essentially a PN junction semiconductor diode that emits a monochromatic (single color) light when operated in a forward biased direction. The basic structure of an LED consists of the die or light emitting semiconductor material, a lead frame where the die is actually placed, and the encapsulation epoxy which surrounds and protects the die. LEDs also are configured to release a large number of photons outward and are housed in a plastic bulb that concentrates the light in a particular direction where most of the light from the diode bounces off the sides of the bulb, traveling on through the outermost end of the bulb.

LEDs of a light emitting device of the invention are arranged in a configuration where the combined arrangement of constituent LEDs 101 are organized to direct emitted radiation onto a tissue or cell target. The arrangement can vary depending on the target area and can range from one or a few LEDs for directing radiation onto a small or focused area to several rows and columns of 5-10 or more LEDs sufficient to illuminate a broad area such as the scalp. Substantially larger target areas can be illuminated by including in the arrangement more individual LED elements to comprise the unit array or by combining multiple array units until a desired array size is produce. Because of the modular nature of LEDs as a constituent element of one or more array units, a light emitting device of the invention can have an arrangement of LEDs consisting of any size, shape or other configuration particularly useful for a desired application. An exemplary arrangement of LEDs is shown as an array 100 in FIG. 1 for a light emitting device suitable for illuminating the scalp for treatment of follicular cell apoptosis and alopecia. This exemplary alopecia-type arrangement, shown in FIG. 1, consists of a circular shape having 96 LEDs configured in rows and columns that illuminate a portion of the scalp area of an adult head.

The LED arrangement 100 shown in FIG. 1 exemplifies a two dimensional array configuration having dimensions of about 120×140 mm². However, a light emitting device of the invention also can have other configurations including, for example, three dimensional and higher orders of complexity for the arrangement of constituent LED elements. For example, FIG. 1 shows a two dimensional planar array for illuminating an area of about 140×160 mm² of the scalp or other tissue area to increase vaso perfusion and cellular energy levels and to promote cellular regeneration or re-growth.

Array configurations 100 of a light emitting device of the invention also can include 3 dimensional configurations that, for example, parallel the surface area of a tissue, organ or body structure that will be illuminated. Conforming the array organization to parallel the contours of a target tissue or component, for example, increases the efficiency of illuminating light because more radiation will be focused on non-planar or non-parallel portions of the target tissue. For example, a light emitting device having a configuration for reducing follicle cell apoptosis and useful for the treatment of alopecia can be shaped as a spherical oval or circle so that, at any particular point within the array, emissions from an LED within the array is substantially orthogonal to the plane of the scalp directly below. Similarly, a light emitting device of the invention can be produced to have essentially any desired dimensions, to illuminate a portion or all of the scalp or other target tissue. Moreover, different arrays can be oriented in different directions or different angles to achieve illumination of different planes of a target area or to illuminate different target areas. The device array 100 exemplified in FIG. 1 is of sufficient size to illuminate the crown of an adult scalp. Given the teachings and guidance provided herein, those skilled in the art will know that the dimensions and spatial configuration can be readily adjusted to large or small target areas as well as to planer or more complex target tissue contours to suit a particular application.

The density of constituent LED elements 101 of an array unit should be sufficient to illuminate a target area with near infrared radiation emissions. Generally, near infrared radiation will be uniform over the illuminated target area. However, those skilled in the art will understand that target areas that receive less near infrared radiation compared to other target areas will exhibit a corresponding decrease in rate of treatment. Those skilled in the art will understand that compensatory changes can be made to the treatment for these areas by, for example, increase the length of treatment.

LEDs within the near infrared region of the electromagnetic spectrum will illuminate an area of about 224 cm² when elevated about 1-2 cm above the target area. Increasing or decreasing the distance between LED and target surface correspondingly increases or reduces the area of illumination. For example, increasing the distance from a target will increase the illuminated area but decrease the intensity per unit area. Conversely, decreasing the distance between LED and target will decrease the illuminated area but increase the intensity of radiation impinging on the target per unit area. The relationships of radiation beam length and illuminated area are well known to those skilled in the art.

The light emitting device exemplified in FIG. 1 has a density of about 30 joules/cm². Other useful densities of constituent LED elements within an array of the invention include, for example, between 10-50 joules/cm², preferably between 20-40 joules/cm² and more preferably between 25-35 joules/cm². Given the teachings and guidance provided herein, those skilled in the art will know that any of a variety of LED densities can be used in a light emitting device of the invention to suit a particular need. In addition to the densities exemplified above, other useful LED densities of an array of the invention include, for example, all integer values between each of the above ranges as well as between about 5-55 joules/cm² or more.

LED constituents of a light emitting device of the invention emit near infrared radiation having wavelengths between about 600-1000 nm of the electromagnetic spectrum. LEDs emitting some or all of the wavelengths within this range can be used in an array of the device. Moreover, two or more LEDs within the array can emit the same near infrared wavelength or different near infrared wavelengths. Thus, an array can contain a plurality of LEDs where each LED emits the same near infrared wavelength, a plurality of LEDs where each LED emits a different near infrared wavelength or a plurality of LEDs where two or more LEDs emit the same near infrared wavelength and two or more different LEDs emit the same near infrared wavelength that differ from the first plurality of LEDs. Accordingly, an array can contain a plurality of LEDs emitting the same near infrared wavelength or contain a plurality of LEDs that together emit a plurality of different near infrared wavelengths.

For example, an array can contain pluralities of, for example, 2, 3, 4, 5, 6, 7, 8, 9 or 10 or more LEDs that emit the same near infrared wavelength. Arrays containing larger pluralities of the same near infrared wavelength, including combinations with pluralities of different wavelengths can include, for example, a plurality of 15, 20, 25, 50, 75 or 100 or more LEDs that emit the same near infrared wavelength. These LED pluralities, corresponding to a set that emits the same near infrared wavelength, can be combined with other sets emitting different near infrared wavelengths to constitute an array of a light emitting device of the invention. For arrays containing a large number of LED sets, the number and size of the sets can be adjusted to the size and density of the array and the desired application of the light emitting device of the invention. The array 100 exemplified in FIG. 1 contains a plurality of 96 LEDs corresponding to 12 different near infrared wavelengths. The wavelengths are indicated adjacent to each LED and the specifications for the plurality of LEDs are shown below in Table 1. Therefore, on average, the array exemplified in FIG. 1 contains about 8 LED sets that emit the same near infrared frequency. The exact number of LEDs per wavelength is shown in the box inset in FIG. 1. Other sizes of arrays useful for various different treatments including, for example, decreasing follicular apoptosis include pluralities of about 80-120 LEDs, generally about 50-150 LEDs and more generally about 30-200 LEDs.

Near infrared emissions from an light emitting device of the invention can range from about 600 nm to about 1000 nm. One range of near infrared radiation that beneficially induces the production of nitric oxide (NO), facilitating healing, and excites mitochondrial chromophores, enhancing cellular energy levels consists of any near infrared radiation within the range between about 610 nm to about 980 nm. Illuminating follicle cells of a target tissue such as the scalp with wavelengths spanning this range of near infrared radiation will synergistically combine to decrease follicular apoptosis. Other ranges of near infrared wavelengths having similar synergistic effects include, for example, a plurality of near infrared wavelengths between about 640 nm to about 950 nm or a plurality of near infrared wavelengths between about 680 nm to about 910 nm.

The distribution of near infrared wavelengths within the ranges exemplified above can vary but generally will correspond to increments of 5-40 nm differences per LED or other illumination source. A particularly useful increment for wavelength distributions is about 20 nm per LED or other near infrared light source. However, any increment that can distribute all near infrared wavelengths across a selected range can be employed in a light emitting device of the invention. For example, the device exemplified in FIG. 1, having a circular arrangement, utilizes LEDs having 20-30 nm wavelength intervals ranging from 680 nm to 910 nm. Other distributions and arrangements also can be employed in a near infrared light emitting device of the invention. Given the teachings and guidance provided herein, those skilled in the art will understand that various combinations and permutations of near infrared wavelengths, the increment of wavelength to use between, for example, component LED illumination sources and the pattern or arrangement of differing near infrared wavelengths can be produced that will synergistically combine to effect a decrease in follicular apoptosis

For example, near infrared wavelengths within the range of about 890 nm beneficially promote NO production. Near infrared wavelengths within the range of about 680-910 nm beneficially excite mitochondrial energy levels. Arrays containing both near infrared wavelengths will generate the combined effects of inducting NO production and increasing mitochondrial energy states. A simple array of a device of the invention can have, for example, one near infrared illumination source in the lower wavelength range and one near infrared illumination source in the higher wavelength range. Generally, a device will contain a plurality of different wavelengths in each of the lower and higher near infrared ranges, thus increasing near infrared exposure and efficacy of the device for preventing follicle cell apoptosis. In other aspects of the device of the invention, an array can have illumination sources for all or substantially all of the selected range of near infrared wavelengths. In this latter aspect, the wavelength increment per LED or other illumination source would be 1 nm.

The pattern of distribution of near infrared light sources also can vary depending on the need and intended use of a light emitting device of the invention. For example, the pattern exemplified in FIG. 1 shows a approximate even distribution of different wavelengths throughout the circular arrangement. Such an approximate even distribution facilitates uniform exposure of target cells or tissues to substantially all near infrared wavelengths of the device. In other aspects, the pattern of distribution for near infrared light sources can be biased toward particular regions of a target tissue. For example, one area can contain more within one near infrared region compared to another to facilitate relative enhancement of NO production over increased energy states within a predetermined region.

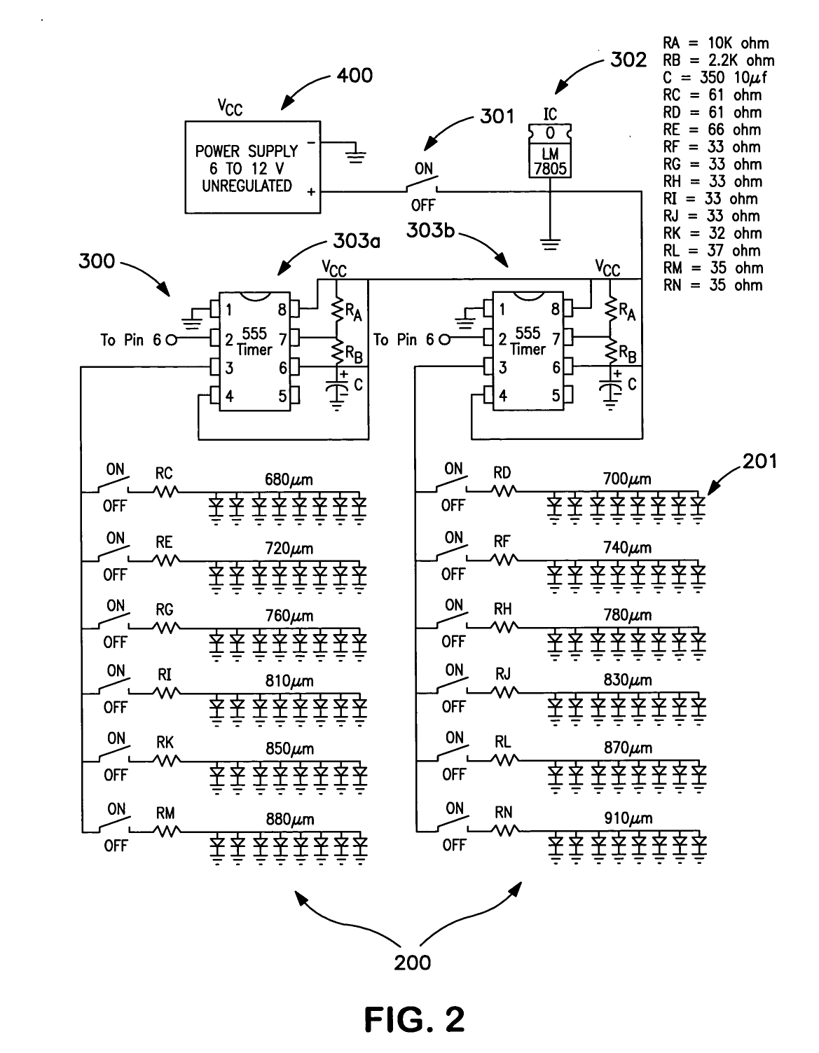

The electrical circuitry 300 and power source 400 can be designed according to well known methods in the electrical engineering art. Exemplified in FIG. 2 is one design that will supply power, regulation and wiring for the light emitting device having 96 near infrared LEDs 201 ranging from 680-910 nm 200 that is shown in FIG. 1. Briefly, the electrical design for the specific device exemplified in FIG. 1 contains a 6-12 volt power supply 400, an on/off switch 301, an integrated circuit 302 and timers 303 operating parallel wiring that supply power to two arrays 200 of LEDs. Each array contains eight LEDs 201 for six different near infrared wavelengths that are arranged in series. The total of 12 different wavelengths for the device exemplified in FIGS. 1 and 2 correspond to 680 nm, 700 nm, 720 nm, 740 nm, 760 nm, 780 nm, 810 nm, 830 nm, 850 nm, 870 nm, 880 nm and 910 nm. Each timer is regulated by resistors R_{A} and R_{B} and a capacitor. Each series of eight LEDs for a particular wavelength is regulated by an on/off switch and a resistor, R_{C} through R_{N}, respectively.

Given the teachings and guidance provided herein, as well as that which is well known in the electrical engineering art, those skilled in the art can design and implement essentially any electrical design to power and regulate one or more illumination arrays, including a large plurality of arrays. Electrical circuitry and regulation is will known in the art and numerous different electrical layouts, formats and power schemes can be readily used in a light emitting device of the invention. For example, the near infrared wavelengths, configuration, density, spatial organization, wavelength distribution and pattern of distribution can be designed to treat a target cell or tissue with a particular pattern of wavelengths, range of wavelengths or both based on the teachings in the application. Once these attributes are selected those skilled in the art can generate an electrical system to power each LED or other illumination source within the array.

The near infrared LEDs or other near infrared illumination source used in a light emitting device of the invention can emit pulsed or continuous radiation or both. Moreover, different LEDs can be configured to emit pulsed or continuous radiation or alternating pulsed and continuous by programming, for example, an integrated circuit, using a computer, other electrical controlling mechanism or by both. Pulsed near infrared radiation is beneficial for decreasing follicle cell apoptosis because it provides a source of overlapping wavelengths. For LEDs exhibiting about 10 cycles per second (sec), durations of near infrared radiation emission ranging from between about 5 msec to continuous emission will beneficially induce NO production and excite mitochondrial chromophores. Other useful durations for pulsed emissions include, for example, between about 50 msec to 1.0 sec as well as durations between about 75-100 msec. A specific example includes a light emitting device applicable for decreasing follicular apoptosis where the LEDs are pulsed at 85 msec or on 0.085/sec using LEDs exhibiting a frequency of 10 cycles per second.

Conversely, durations of no light emission for the above exemplary ranges include, for example, off times of between about 13-18 msec, generally between about 12-20 msec, and more generally between about 1-900 msec. For the specific example of a light emitting device applicable for decreasing follicular apoptosis an off duration can be about 15 msec.

For both light emitting devices applicable to decreasing follicular apoptosis as well as applicable to other treatments, all durations between the exemplified ranges above also can be employed in a light emitting device of the invention. Similarly, all on/off pulse durations above and below the specifically exemplified pulse and off times above for a light emitting device applicable for decreasing follicular apoptosis also can be employed. Therefore, a light emitting device applicable for decreasing follicular apoptosis can have, for example, a pulse time longer or shorter than 85 msec and a corresponding off time shorter or longer than 15 msec, respectively.

For the specific embodiment exemplified in FIG. 2, the length of pulse durations were controlled using the formulas set forth below. Such methods are well known in the art of electrically engineering. Similarly, other methods well known in the art for controlling pulse durations also can be used in a light emitting device of the invention. The nomenclature used below also is well known in the art and correspond to the nomenclature shown in FIG. 2. Briefly, t1 corresponds to charge time or how long an LED is on, which is calculated as 0.693 (R_{A}+R_{B}) C. The term t2 corresponds to discharge time, or how long an LED is off, which is calculated as 0.693(R_{B}) C. T corresponds to the period and equals t1+t2 or 0.693 (R_{A}+2R_{B}) C. Therefore, for the circuit shown in FIG. 2, where the capacitor C is 220 μF, the Frequency is 10 cycles per second.

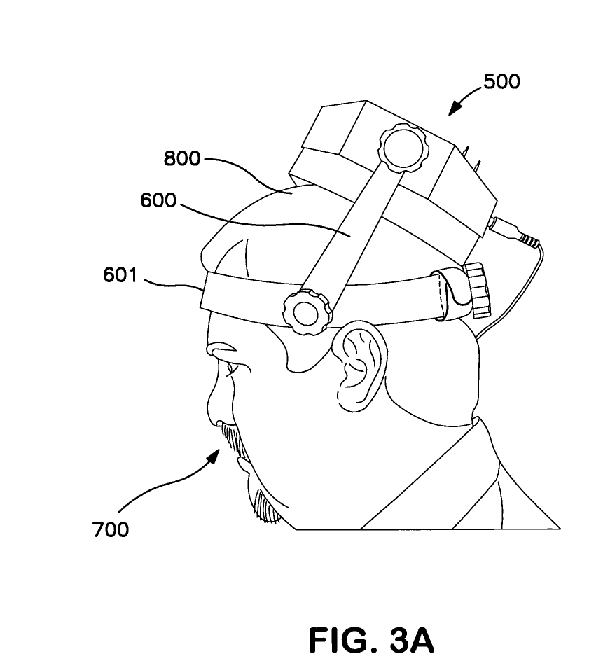

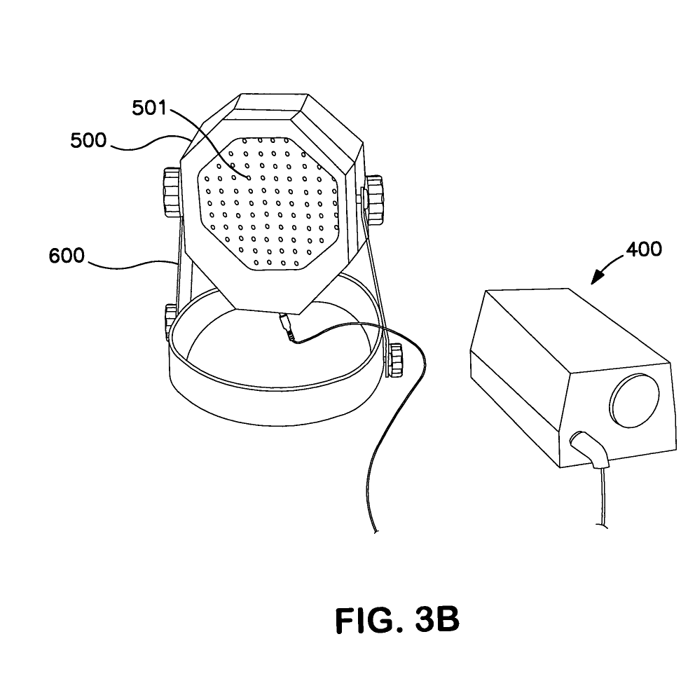

As exemplified in FIG. 3, a light emitting device 500 of the invention can be mounted or affixed to a variety of structures 600 that function to position the near infrared emitting LEDs or other illumination sources over target cells or tissues, including a target area of a subject 700 such as the scalp 800. Positioning of the device on a subject 700 is exemplified in FIG. 3A. Positioning should include orientation of the LEDs shown in FIG. 3B 501 to direct near infrared emissions toward the target cells or tissues. A variety of structures 600 can be used to mount a light emitting device of the invention including permanent mounts to a stationary structure, floor stands, counter stands or permanent or temporary mounts to other apparatuses. Such other apparatuses include, for example, a counter, table, chair, bed or cot suitable for use in a physicians office, clinic or hospital as well as a counter, table, chair, bed or cot suitable for use in the home or office. The structures can be counterbalanced to ensure stability of unit during use.

Given the teachings and guidance provided herein, those skilled in the art will understand that a stationary or temporary mount can be produced for variety of different structures that position and orientate a light emitting device above target cells or tissues given the location of a target area of a subject positioned in an apparatus. In this regard, those skilled in the art will understand that the type of mount, such as a mounting stand or a vertical member that can be used to mount the light emitting device to the apparatus, is chosen to position the device above the target area and the device is affixed to the mount or member to orientate near infrared emissions toward the target. The height, angle and other geometries similarly will be chosen based on the apparatus that will position the subject readied for treatment.

For example, a light emitting device can be mounted to a counter, table, chair or other stationary structure using a vertical member that positions an array of near infrared LEDs in an orientation that directs emissions in a downward direction where the subject is seated and the vertical member elevates the light emitting device above the target area. Conversely, if a subject is in a horizontal position, a stationary mount can be designed to emit near infrared radiation in an upward or horizontal direction to impinge on a target area. Upward directions also can be employed for subjects being treated in a seated position. For the specific example of a device useful for decreasing cellular apoptosis, and particularly for directing near infrared radiation on the scalp for the treatment of alopecia, a seated position can be desirable and a light emitting device can be positioned above the scalp with near infrared emission directed downward over the scalp.

Further, a light emitting device of the invention also can be positioned for directing near infrared radiation to the scalp using a headband 601, as exemplified in FIG. 3. At least one vertical member comprising structure 600 can be used to elevate the light emitting device above the scalp. Generally, two, three or four or more vertical members will be attached to both the headband and the device. The total number of vertical members can vary so long as the device is positioned above the scalp and is stationary. Attachment of the device to the headband can be accomplished by any means well known to those skilled in the art including, for example, injection molding methods, screws, fasteners, and rivets. Attachment is accomplished so that the LEDs or other illumination sources are orientated to direct near infrared emissions downward toward the interior of the headband. Such an orientation will direct near infrared radiation onto the scalp. The lengths of the vertical mounts, stands or other stationary or temporary mounts will be determined based on the distances between near infrared radiation source and target as described previously to ensure effective exposure to the near infrared radiation. A light emitting device of the invention that uses a headband to position the device above the scalp of a subject is exemplified in FIG. 3. Further, a light emitting device can be emlarged to encompass the entire head as long as at least the requisite LED's all have exposure to an area of the scalp being treated.

The invention also provides a method of reducing follicular cell apoptosis. The method includes illuminating a surface area of a scalp containing a follicular hair cell for an effective period of time with an array of light emitting diodes (LED), the LEDs having a plurality of wavelengths within the near infrared region of the electromagnetic spectrum, where the plurality of wavelengths synergistically combine to decrease follicular cell apoptosis.

A light emitting device of the invention can be used to direct a plurality of different wavelengths of near infrared radiation to the surface area of a scalp to decrease follicle cell apoptosis. Reduction in the rate or extent of follicle cell apoptosis will reduce the severity of alopecia. Reduction in the rate or extent of follicle cell apoptosis also will lead to follicle cell regeneration and regrowth with a concomitant reduction in hair loss, stimulation of hair regrowth or both a reduction in hair loss and a stimulation in hair regrowth.

As described previously, exposure of cells including, for example, follicle cells of the scalp, to near infrared radiation within wavelengths between 890 nm induces NO production, facilitating healing of injured or unhealthy cells or tissues. Exposure of cells including, for example, follicle cells of the scalp, to near infrared radiation within wavelengths between 680-910 nm excites mitochondrial chromophores, which leads to enhanced cellular energy levels, cellular regeneration and cellular regrowth. The method of the invention combines the above two near infrared ranges of wavelengths to effect the reduction in follicular cell apoptosis. The combination of the above lower range near infrared wavelengths and the above higher range near infrared wavelengths yields a synergistic effect for decreasing follicular cell apoptosis compared to that which can be observed for either NO production or chromophore excitation alone or compared to that expected for the additive effect for the combination of NO production and chromophore excitation. This synergistic effect can be beneficially employed to efficaciously decrease follicular cell apoptosis for the treatment of alopecia.

The method of the invention includes illuminating a surface area of a scalp containing a follicular hair cell with a plurality of near infrared wavelengths between about 600-1000 nm. The near infrared light emitting device describe previously can be used in the method of reducing follicular cell apoptosis. Accordingly, near infrared wavelengths ranging from about 680-910 nm, generally about 640-950 nm and more generally about 610-980 nm can employed. The illumination sources can be LEDs or other near infrared illumination sources and they can be configured for pulsed or continuous exposure. The array of LEDs, for example, can range from about 80-120, generally about 50-15 and more generally about 30-200 different members and can be positioned above the scalp using a stationary mount or a headband. An exemplary device useful in the method of the invention is shown in FIGS. 1-3.

Exposure of a scalp area to near infrared radiation within the wavelengths described above and previously produces a synergistic combination of near infrared radiation that decreases follicular cell apoptosis. The scalp or other follicular cell target area, for example, is exposed to the plurality of near infrared radiation wavelengths of the invention for an effective period of time. An effective period of time can vary depending, for example, on the age of the subject, the extent of cellular apoptosis and sensitivity of the skin. For example, aging subjects can respond slower to treatment and correspondingly longer treatment periods can be used to achieve the same result as with a younger subject. Similarly, the presence of more extensive or active cellular apoptosis also can require longer treatment periods in order to initially overcome or reverse the progression of the damaged tissue. Sensitive skin can be treated for shorter periods of time to avoid unnecessary stress and therefore prevent symptoms associated with sensitive skin. An increase in treatment frequency can compensate for the shorter exposure times so as to maintain an overall effective period of time exposure to the plurality of near infrared wavelengths of the invention.

Effective periods of time of exposure or illumination by the near infrared radiation of the invention can range, for example, from about 3 minutes to 45 minutes or longer. Effective periods of time between about 5-30 minutes are particularly useful for inducing a synergistic effect that decreases follicular cell apoptosis. Such exposure times will increase NO production and excite mitochondrial energy states to increase the overall health of the exposed cell or tissue. Other useful effective periods of time include, for example, exposures to a plurality of different near infrared wavelengths of the invention of between about 10-20 minutes and particularly between about 12-18 minutes. An effective period of time of 15 minutes has been found to promote follicular cell regeneration and regrowth with concomitant hair regrowth. Therefore, the method of the invention is therapeutically effective for treating alopecia to prevent further hair loss and/or to regrow previously lost hair.

All time periods between the above exemplified ranges also can be employed in the method of the invention. Given the teachings and guidance provided herein, those skilled in the art will understand that effective periods of time can be adjusted to achieve a desired outcome. For example, longer exposure times within the above ranges will result in greater efficacy at reducing follicular cell apoptosis. Conversely, shorter periods of exposure will decrease efficacy per treatment but will result in less undesirable effects that may be caused by exposure to near infrared radiation.

In addition to effective periods of time for near infrared exposure, various exposure regimens can be implemented to accomplish an overall treatment plan. For example, the method of the invention can be employed once, twice or three or more times to sequentially increase efficacy of the treatment. A particularly useful exposure regimen can be illumination of a target scalp surface two or more times a week for a period of one or more months. Moreover, those skilled in the art will understand given the teachings and guidance provided herein that various combinations of different effective periods of time together with different exposure regimens can be employed to achieve the same or similar result. For example, shorter effective periods of time can be employed in combination with increased frequency of exposure regimen to achieve a similar result as that obtained with longer effective periods of time using an exposure regimen of having a decreased frequency. Accordingly, changes in effective periods of time can be compensated for by correspondingly adjusting the exposure regimen up or down. An effective exposure regimen of three times a week employing an effective period of time of 15 minutes has been found to regrow lost scalp hair. Such results can be achieved where the treatment continues for between about 1-3 months. Therefore, the method of the invention is therapeutically effective for treating alopecia by reversing hair loss and inducing hair regrowth.

TABLE 1
LED CHARACTERISTICS

| | | | | Other |
|---|---|---|---|---|
| No. of | | Wave- | | Character- |
| LEDs | Pat Id No. | length | Power | istics |
| 8 LEDs | ELD-910-535 | 910 nm | 32 mW | 5 mm clear |
| | | | | epoxy |
| 8 LEDs | ELD-680-524 | 680 nm | | 5 mm clear |
| | | | | epoxy |
| 8 LEDs | LED-700-02AU, | 700 nm | | 5 mm epoxy |
| | RED LED | | | |
| 8 LEDs | ELD-720-524 | | | |
| 8 LEDs | LED740-01AU | 740 nm | | 5 mm clear |
| | | | | epoxy |
| 8 LEDs | LED760-40M32 | 760 nm | 10 mW at 50 mA | 20 deg., |
| | | | | TO-1 |
| 8 LEDs | ELD-780-524, | | | 5 mm, epoxy |
| | IR-LED | | | |
| 8 LEDs | ELD-810-525 | 810 nm | 28 mW at 100 mA | 5 mm |
| 8 LEDs | LED830-03AU, | 830 nm | | 5 mm epoxy |
| | IR-LED | | | |
| 8 LEDs | LED850-04UP | 850 nm | 22 mW at 50 mA | 5 mm epoxy |
| 8 LEDs | ELD-870f-515-2 | 870 nm | 16 mW at 100 mA | |
| 8 LEDs | OPE5687HP | 880 nm | 45 mW at 100 mA | 22 deg |

It is understood that modifications which do not substantially affect the activity of the various embodiments of this invention are also included within the definition of the invention provided herein. Accordingly, specific examples described herein are intended to illustrate but not limit the present invention.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A light emitting device, comprising an array of light emitting diodes (LED), said array being arranged to illuminate a surface area of a scalp, said LEDs having a plurality of wavelengths within the near infrared region of the electromagnetic spectrum between about 600-1000 nm, wherein said plurality of wavelengths synergistically combine to decrease follicular apoptosis.

2. The device of claim 1, wherein said plurality of wavelengths comprise a range between about 610 nm to about 980 nm.

3. The device of claim 2, wherein said plurality of wavelengths comprise a range between about 640 nm to about 950 nm.

4. The device of claim 1, wherein said plurality of wavelengths comprise a range between about 680 nm to about 910 nm.

5. The device of claim 1, wherein said array of light emitting diodes further comprises LEDs that are pulsed.

6. The device of claim 5, wherein said pulsed LEDs comprise a duration of light emission of between about 5 msec to continuous duration, preferably between about 50 msec to 1 sec, more preferably between about 75-100 msec.

7. The device of claim 5, wherein said pulsed LEDs comprise a duration of light emission of about 85 msec.

8. The device of claim 5, wherein said pulsed LEDs comprise a duration of no light emission of between about 1-900 msec, preferably between about 12-20 msec, more preferably between about 13-18 msec.

9. The device of claim 5, wherein said pulsed LEDs comprise a duration of no light emission of about 15 msec.

10. The device of claim 1, wherein said array of light emitting diodes comprises about 30-200, preferably about 50-150, and more preferably about 80-120 LEDs.

11. The device of claim 1, wherein said array of light emitting diodes comprises the an arrangement of LEDs corresponding to the arrangement of LEDs shown in FIG. 1.

12. The device of claim 1, further comprising a headband, said headband being attached to said device through at least one vertical member, wherein said vertical member positions said array of light emitting diodes above said headband in an origination to direct light emitted from said LEDs toward said headband.

13. The device of claim 1, further comprising a stationary mount, said stationary mount being attached to said device through at least one vertical member, wherein said vertical member positions said array of light emitting diodes above said mount in an origination to direct light emitted from said LEDs in a downward direction.

14. A method of reducing follicular cell apoptosis, comprising illuminating a surface area of a scalp containing a follicular hair cell for an effective period of time with an array of light emitting diodes (LED), said LEDs having a plurality of wavelengths within the near infrared region of the electromagnetic spectrum between about 600-1000 nm, wherein said plurality of wavelengths synergistically combine to decrease follicular cell apoptosis.

15. The method of claim 14, further comprising an increase in nitric oxide production compared to an untreated follicular hair cell.

16. The method of claim 14, further comprising mitochondrial chromophore activation.

17. The method of claim 14, wherein said effective period of time comprises about 5-30 minutes, preferably about 10-20 minutes or more preferably about 12-18 minutes.

18. The method of claim 14, wherein said effective period of time comprises about 15 minutes.

19. The method of claims 17 or 18 wherein said effective period of time further comprises two or more times a week.

20. The method of claim 14, further comprising promoting vaso perfusion of said scalp.

21. The method of claim 14, wherein said decrease in follicular apoptosis reduces hair loss.

22. The method of claim 14, wherein said plurality of wavelengths comprise a range between about 610 nm to about 980 nm.

23. The method of claim 22, wherein said plurality of wavelengths comprise a range between about 640 nm to about 950 nm.

24. The method of claim 14, wherein said plurality of wavelengths comprise a range between about 680 nm to about 910 nm.

25. The method of claim 14, wherein said array of light emitting diodes further comprises LEDs that are pulsed.

26. The method of claim 25, wherein said pulsed LEDs comprise a duration of light emission of between about 5 msec to continuous duration, preferably between about 50 msec to 1 sec, more preferably between about 75-100 msec.

27. The method of claim 25, wherein said pulsed LEDs comprise a duration of light emission of about 85 msec.

28. The method of claim 25, wherein said pulsed LEDs comprise a duration of no light emission of between about 1-900 msec, preferably between about 12-20 msec, more preferably between about 13-18 msec.

29. The method of claim 25, wherein said pulsed LEDs comprise a duration of no light emission of about 15 msec.

30. The method of claim 14, wherein said array of light emitting diodes comprises about 30-200, preferably about 50-150, and more preferably about 80-120 LEDs.
